(19) United States
(12) Patent Application Publication
(10) Pub. No.: US 2006/0135453 A1
(43) Pub. Date: Jun. 22, 2006

(54) Down-regulation of target-gene with pei/single-stranded oligoribonucleotide complexes

(76) Inventors: Jean-Charles Bologna, Nimes (FR); Jonathan Hall, Dornach (CH); Francois Jean-Charles Natt, Aesch (CH); Jan Weiler, Lorrach-Haagen (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/543,362

(22) PCT Filed: Jan. 30, 2004
(86) PCT No.: PCT/EP04/00897

§ 371 Date: Jan. 19, 2006

Related U.S. Application Data
(60) Provisional application No. 60/443,977, filed on Jan. 31, 2003.

(51) Int. Cl. A61K 48/00

(52) U.S. Cl. 514/44

(57) ABSTRACT

The present invention provides methods for the downregulation of target genes by an RNA interference mechanism using short single stranded RNA and a cationic polymer, such as linear PEI.

### FIELD OF THE INVENTION

The present invention relates to methods for reducing the expression of target genes using a cationic polymer and single-stranded ribonucleotide oligomers.

### BACKGROUND OF THE INVENTION

mRNA knock-down reagents such as antisense oligonucleotides (ASOs) or duplexes of short RNAs, also known as small interfering RNAs (siRNAs), have become powerful tools in modulating the expression of genes and thereby contributing to the elucidation of their function and putative role in disease processes.

Initially, the most common approach to achieve gene-specific inhibition was antisense technology, wherein single-stranded nucleic acid (oligodeoxynucleotide or oligoribonucleotide) complementary to the messenger RNA of the gene of interest is introduced into the cell (Thompson, 2002). More recently, duplexes of short RNAs, also known as small interfering RNAs (siRNAs), have been demonstrated to efficiently inhibit gene expression upon cellular delivery with an appropriate transfection reagent by a mechanism called RNA interference (Fire et al., 1998) in mammalian cells (Elbashir et al., 2001). siRNAs are formed by two complementary strands of RNA forming a 19-21 nucleotides double-stranded region and where each of the strand bears a 1-3 nucleotides overhang. RNA interference has been described as a naturally occurring defense mechanism against viral dsRNA. The proposed mechanism of action suggests the unwinding of the double-stranded siRNA followed by formation of ssRNA-enzymatic complexes as intermediates in the gene silencing process, thereby blurring the distinction between RNAi and antisense effects (Martinez et al. 2002, Schwarz et al. 2002).

However, there are considerable limitations with oligonucleotide-based approaches relating to delivery, stability, and dose requirements. Unmodified phosphodiester oligonucleotides, and more particularly oligoribonucleotides, are highly sensitive towards nuclease degradation and in general, spontaneous uptake of nucleic acids is extremely inefficient. As a consequence, much of the effort in developing oligonucleotide technology has been focused on the production of transfection reagents enhancing the cellular uptake and on the synthesis of modified nucleic acids that are both stable to nuclease digestion and able to diffuse readily into cells. In the past decade, analogues of phosphodiester linkage and novel chemically modified nucleoside building blocks such as 2′-O-MOE (methoxyethyl) derivatives (Martin et al, 1995) have been designed that have significantly improved stability, potency and selectivity of traditional phosphodiester and phosphorothioate antisense oligomers.

Gene silencing in mammalian cells using unmodified ssRNA via a RNAi mechanism has been recently demonstrated in HeLa cells (Martinez et al., 2002, Schwarz et al. 2002). However, the efficiency of ssRNA was low as compared to dsRNA. Furthermore, HeLa cells are known to be poor in nucleases. Thus, the approach of Martinez and Schwarz is not generally applicable, as, for instance, it cannot be applied to other cell lines with more nuclease activity. For instance, ssRNAs used in other mammalian cell-lines, such as H-9, MOLT-3 or T-24 cells, and supposed to act via an antisense mechanism, had to be fully modified to elicit mRNA degradation (Agrawal et al., 1992, Wu et al., 1998). The chemical modifications used to stabilize ssRNA may have negative effects depending on the regulation mechanism involved. More specifically, modified ssRNAs might allow mRNA degradation through an antisense mechanism but might probably have a lower affinity with enzymatic complexes involved in other down-regulation pathways, such as RISC-complex formation induced in the RNA interference pathway. By contrast, chemical modifications applied on the 3′-end of RNA duplexes have a minimal or no influence on the silencing activity (Schwarz et al. 2002) However, there is a clear need of a general applicable method using unmodified ssRNA for RNAi, because such an approach would be clearly advantageous in terms of cost of reagents but is limited in its potency because of the poor stability of ssRNA as compared to dsRNA. Because of their analogy with single strands RNA bearing 3′-modified overhangs involved in a siRNA duplex, minimally 3′-modified phosphodiester single strand RNA, able to act in the RNAi pathway, might also act as antisense knock-down reagents. Thus, it would be clearly desirable to have a method allowing the use of 3′-minimally modified single strand RNA for the down-regulation of specific target genes.

The present invention now provides a method that allows the down-regulation of specific target genes in mammalian cells by the application of minimally modified ssRNA in combination with a cationic polymer. The present invention thus provides for the first time efficient use of ssRNA for RNA interference as gene inhibitors and is in particular useful for high-throughput screening.

### SUMMARY OF THE INVENTION

The present invention relates to the knock-down of target genes using ssRNA and a cationic polymer such as PEI.

In a first aspect, the invention provides a method for reducing the expression of a target gene comprising exposing a cell to a single-stranded oligoribonucleotide and PEI, wherein said single-stranded oligoribonucleotide comprises a region of less than 50, preferably less than 25 nucleotides complementary to the mRNA encoded by said target gene. In another preferred embodiment, the complementary region is from 10 to 30, more preferably from 15 to 25, in a particularly preferred embodiment from 19 to 21 nucleotides. In another preferred embodiment, the complementary region is 16, 17, 18, 19, 20, 21, 22, 23, 24 or 25 nucleotides to the mRNA encoded by said target gene.

In accordance with a preferred embodiment of the present invention, said cells are, eukaryotic cells, more preferably mammalian cells, most preferably human cells. Said PEI is preferably linear PEI and the N/P ratio is preferably between 2 and 10, more preferably between 3 and 8.

In accordance with another embodiment of the present invention, said ssRNA comprises 1, 2, 3, or 4 mismatches. In one embodiment, the mismatches are contiguous.

In accordance with another embodiment of the present invention, said single-stranded oligoribonucleotide comprises 1 to 10, preferably 1 to 8, more preferably 1 to 6 chemically modified ribonucleotide residues. Particularly preferred are oligoribonucleotides with 1, 2, 3, 4, or 5 chemically modified residues. Preferred chemical modifications are 2′-O-MOE modifications or modifications in the internucleosidic backbone such as for instance phosphorothioate.

Said target gene is, in accordance with a preferred embodiment of the present invention, a human gene. Preferably, the gene is overexpressed in a pathological condition, more preferably the gene is an oncogene, cytokine gene, viral gene, bacterial gene, development gene or prion gene.

According to a related aspect of the present invention, said methods are provided, wherein the target gene is downregulated by RNA interference.

In another related aspect, the present invention provides ssRNA and PEI for RNA interference.

In another aspect, the present invention provides a kit comprising ssRNA and PEI in an amount sufficient to inhibit the expression of a target gene, wherein said ssRNA comprises a region complementary to the target gene.

### BRIEF DESCRIPTION OF THE DRAWINGS

FIG. 1 shows inhibition of P2X3 mRNA by linear PEI-mediated transfection of P2X3 single strand at 400 nM.

FIG. 2 shows Inhibition of P2X3 mRNA by linear PEI-mediated transfection of P2X3 single strand at 200 nM.

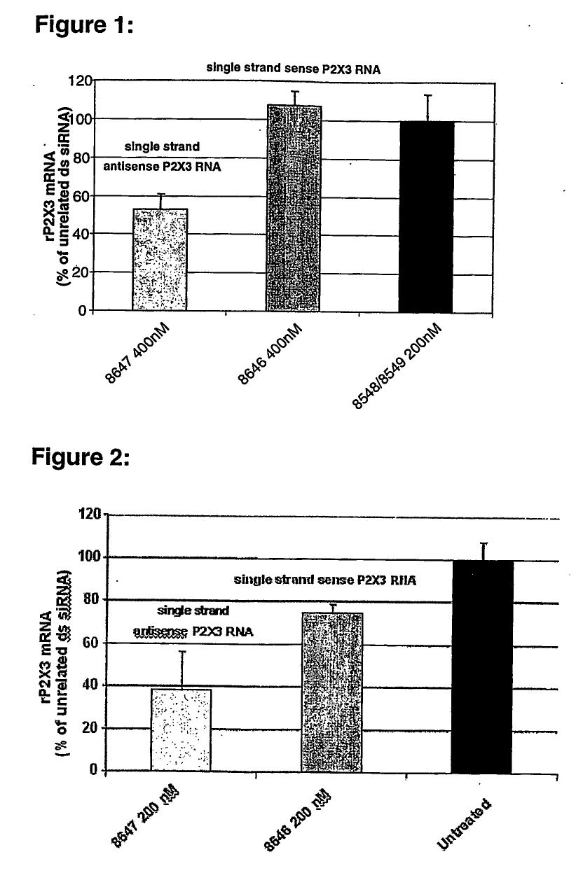

### DETAILED DESCRIPTION OF THE INVENTION

Abbreviations

ASO Antisense oligonucleotide

CHO Chinese hamster ovarian cell line

FBS fetal bovine serum

MEM Minimal essential medium

ON oligonucleotide

RT-Q-PCR real-time quantitative reverse-transcriptase PCR

(I) PEI (linear) Polyethylenimine

PBS Phosphate Buffer Saline

RNAi RNA interference

siRNA Small-interfering RNA

TOM 2′-O-[(triisopropylsilyl)oxy]methyl

2′-O-MOE 2′-O-methoxyethyl

2′-O-Me 2′-O-methyl

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Harlow, E. and Lane, eds., 1988, “Antibodies: A Laboratory Manual”, Cold Spring Harbor Press, Cold Spring Harbor, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

As used herein and in the appended claims, the singular forms “a”, “an”, and “the” include plural reference unless the context clearly dictates otherwise.

For the purpose of the present invention “ssRNA” or “oligoribonucleotide” refer interchangeably to a single-stranded ribonucleotide oligomer as commonly defined and understood in the art.

“Chemical modifications” or “modifications”, in accordance with the present invention, include all alterations of the ribonucleoside oligomers by chemical means such as for instance addition or removal of a chemical moiety or replacement of one chemical moiety with another chemical moiety. In particular, the replacement of non-bridging oxygen atoms by sulfur atoms in internucleosidic bonds and the addition of substituent to the 2′-OH group of the sugar unit are included in the term chemical modification.

“RNA interference” is a common term of the art. Assays allowing to measure downregulation of a target gene by RNA interference or to determine if downregulation of a target gene occurs via an RNA interference mechanism are known in the art, such assays are described for instance in Caplen et al, 2001; Elbashir et al., 2001, D. Husken et al, 2003.

The present inventors have successfully down-regulated the expression of target genes using ssRNA and a linear cationic polymer. So far, unmodified ssRNA has been limited as mRNA knock-down reagent due to its high nuclease sensitivity. ssRNA stabilization has normally been achieved by chemical modifications, such as replacement of all phosphodiester linkages by phosphorothioate linkages (Agrawal et al., 1992, Wu et al., 1998) or by using chimeric compounds, for example bearing 2′-OMe wings (8 to 12 2′-modified ribonucleotides) and a minimum gap of 5 to 9 phosphorothioate modifications to induce mRNA degradation (Wu et al., 1998). The inventors have discovered in accordance with the present invention that by using a cationic polymer, in particular linear PEI, minimally modified ssRNA can be efficiently transfected and stabilized such that the use of phosphodiester ssRNA for mRNA knock-down reagents becomes feasible. Thus, the use of such a cationic transfection reagent overcomes the need of extensive chemical modifications of ssRNA used for the down-regulation of target genes and allows for the first time the application of phosphodiester ssRNA for this purpose.

In a first aspect, the present invention provides a method for the down-regulation of a target gene by exposing a cell to ssRNA and a cationic polymer. Said cationic polypeptides include but is not limited to poly-lysines, poly-arginines, poly-histidines, polylactides and co-polymers of lactic acid and glycolic acid (P(LA-GA)), polysaccharides (DEAE-dextran. In a particularly preferred embodiment the cationic polymer is polyethylenimine (PEI), more preferably linear PEI.

The PEI used in accordance with the presence invention is preferably linear PEI with a molecular weight of 100 to 1,000,000 daltons, more preferably 500 to 200,000 daltons or 1,000 to 100,000. The PEI may be further modified, for instance by hydrophilic polymers such as polyethyleneglycol (PEG). Various types of PEI are commercially available, for instance from Aldrich or Bayer. There are also methods for the production of suitable PEI reagents known in the art, for instance in Fischer et al. 1999.

Polyethylenimine (PEI) is a cationic polymer of ethylenimine exhibiting the highest positive charge density when fully protonated in aqueous solution. Every third atom is an amino nitrogen that can be protonated (Boussif, O. et al., 1995, Behr, J. P., 1997). Branched PEIs contain primary, secondary and tertiary amino groups with different degree of branching, thereby protonable at various pH, whereas linear PEIs contain mainly or exclusively secondary amino groups. Linear PEIs are low molecular weight polymers, generally around 20000-25000 Da. The structure of the commercially linear PEI JetPEI is: HO(CH₂)₂—(CH₂—CH₂—NH)n-(CH₂)₂—OH

Thus, in one embodiment, the present invention provides the use of PEI, in particular linear PEI, and ssRNA, which is preferably unmodified or minimally modified, for the downregulation of a target gene.

The amount of ssRNA necessary for the downregulation of a target gene may be determined empirically and is within the skill of a person skilled in the art. The amount of the cationic polymer depends on the amount of ssRNA used. For PEI, for instance, the ratio of the number of total nitrogen atoms of PEI to the number of phosphate groups of the ssRNA (N/P ratio) is a suitable parameter for determining the amount of PEI to efficiently deliver a given amount of ssRNA. In a preferred embodiment the N/P ratio is from 2 to 10, more preferably from 3 to 8. A particularly preferred ratio is 5. Preferred ratios are the necessary amount of linear PEI (i.e. amount of nitrogen atoms) to efficiently complex the oligonucleotide, to allow an uptake and release of the complex from the endosomes after uptake. At a certain concentration of ssRNA, there might be enough PEI to elicit an efficient uptake but not to induce an effective release from the endosomes (particularly at lower ssRNA concentrations).

A variety of chemical modifications of ribonucleotides and oligoribonucleotides which are useful for antisense technology are known in the art (see for instance: Freier S. M. and Altmann K. H., 1997). Whereas in a particularly preferred embodiment, the ssRNA predominantly consists of unmodified ribonucleotides, the present invention also envisages the use of ssRNA which contains some chemical modifications. Preferred chemically modified ssRNA in accordance with the present invention comprise 1 to 10, preferably 1 to 5 synthetic ribonucleotide analogues comprising a modification of the 2′-OH group, in particular a 2′-O-alkyl group, or 1 to 10, preferably 1 to 5 synthetic deoxyribonucleotides, or any modification at the 3′-end hydroxylic function. In a more preferred embodiment the modifications are 2′-OMe, 2′-O-MOE. Whereas ssRNA containing phosphodiester internucleosidic bonds is preferred, a limited number of internucleosidic bonds may be chemically modified. Thus, in another preferred embodiment of the present invention, the ssRNA comprises 1 to 10, preferably 1 to 5 modifications of the phosphodiester backbone, such as for example phosphorothioate, phosphorodithioate, boranophosphate, phosphoroselenoate, phosphorodiselenoate, phosphoroanilothioate, phosphoranilidate, phosphoramidate, peptide, and the like linkages. In another preferred embodiment, the modifications are located at the 5′ and/or at the 3′ end of the ssRNA molecule. In yet another preferred embodiment of the invention, the ssRNA contains a 5′ phosphate. However, the ssRNA may comprise in addition or alternatively other of the numerous modifications known in the art (Freier S. M. and Altmann K. H., 1997), the number of modified residues more preferably being from 1 to 5.

In another preferred embodiment, the ssRNA comprises one or more deoxyribonucleotides. Preferred is a stretch of 1 to 10, preferably 1 to 5 deoxyribonucleotides, possibly flanked on one or both sides by stretches of ribonucleotides, preferably on the 3′-end.

The ssRNA comprises a region of less than 50 nucleotides and preferably more than 15 nucleotides that is complementary to a given target gene to be down-regulated. More preferred are lengths of 16, 17, 18, 19, 20, 21, 22, 23, 24 or 25 nucleotides. In a particularly preferred embodiment, the ssRNA consists of the region complementary to a given target gene. In another embodiment of the invention, the complementary regions contains 1, 2, 3 or 4 mismatches.

Every cell that is transfectable by cationic polymers, in particular by PEI, can be used for the present invention. Preferred cells are eukaryotic cells, mammalian cells, more preferred are rodent and particularly preferred are human cells. The cells may be derived from various tissues, they include without limitations for instance cells from the inner cell mass, extraembryonic ectoderm or embryonic stem cells, totipotent or pluripotent, dividing or non-dividing, parenchyma or epithelium, immortalized or transformed, or the like. The cell may be a stem cell or a differentiated cell. Cell types that are differentiated include without limitation adipocytes, fibroblasts, myocytes, cardiomyocytes, endothelium, dendritic cells, neurons, glia, mast cells, blood cells and leukocytes (e.g., erythrocytes, megakaryotes, lymphocytes, such as B, T and natural killer cells, macrophages, neutrophils, eosinophils, basophils, platelets, granulocytes), epithelial cells, keratinocytes, chondrocytes, osteoblasts, osteoclasts, hepatocytes, and cells of the endocrine or exocrine glands, as well as sensory cells.

The ssRNA may be synthesized either by chemical methods, which are well established in the art, or by biological methods such as, for instance, by in vitro transcription using a cellular RNA polymerase or a bacteriophage RNA polymerase (e.g., T3, T7, SP6).

As disclosed herein, the present invention is not limited to any type of target gene or nucleotide sequence. For example, the target gene can be a cellular gene, an endogenous gene, an oncogene, a transgene, or a viral gene including translated and non-translated RNAs. The following classes of possible target genes are listed for illustrative purposes only and are not to be interpreted as limiting: transcription factors and developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, ERBB2, ETSI, ETV6, FGR, FOS, FYN, HCR, HRAS, JUN, KRAS, LCK, LYN, MDM2, MLL, MYB, MYC, MYCLI, MYCN, NRAS, PIMI, PML, RET, SKP2, SRC, TALI, TCL3, and YES); tumor suppressor genes (e.g., APC, BRAI, BRCA2, CTMP, MADH4, MCC, NFI, NF2, RBI, TP53, and WTI); and enzymes (e.g., ACP desaturases and hydroxylases, ADP-glucose pyrophorylases, ATPases, alcohol dehydrogenases, amylases, amyloglucosidases, catalases, cyclooxygenases, decarboxylases, dextrinases, DNA and RNA polymerases, galactosidases, glucose oxidases, GTPases, helicases, integrases, insulinases, invertases, isomerases, kinases, lactases, lipases, lipoxygenases, lysozymes, peroxidases, phosphatases, phospholipases, phosphorylases, proteinases and peptidases, recombinases, reverse transcriptases, telomerase, including RNA and/or protein components, and topoisomerases).

The ssRNA preferably comprises a region complementary to one single gene, but may also contain more than one region which are complementary to more than one gene. Also envisaged are methods in which the cells are exposed to several species of ssRNA, which comprise regions complementary to different genes.

By inhibiting enzymes at one or more points in a metabolic pathway or genes involved in pathogenesis, the effect may be enhanced: each activity will be affected and the effects may be magnified by targeting multiple different components. Metabolism may also be manipulated by inhibiting feedback control in the pathway or production of unwanted metabolic byproducts.

The cells can be exposed to ssRNA and PEI in vitro or ex vivo and then subsequently placed into an animal to affect therapy, or the ssRNA and PEI can be directly administered in vivo. A method of gene therapy can therefore be envisioned, typically by introducing a ssRNA specific for a target gene in the presence of PEI into a cell. Any target gene known to cause the disease or condition needing treatment can be used. For example, tumor cells can be targeted using homing viral vectors, tumor-specific promoters or by designing ssRNA molecules effective in inhibiting tumor-specific genes (e.g., telomerase) and oncogenes. Treatment includes amelioration or avoidance of any symptom associated with the disease or clinical indication associated with the pathology, and this may include prophylactic therapy. A further preferred embodiment relates to administering to a subject ES cells treated with ssRNA and PEI to inhibit a desired target gene.

A gene derived from any pathogen may be targeted for inhibition. For example, the gene could cause immunosuppression of the host directly or be essential for replication of the pathogen, transmission of the pathogen, or maintenance of the infection. Cells at risk for infection by a pathogen or already infected cells, such as cells infected by human immunodeficiency virus (HIV) infections, influenza infections, malaria, hepatitis, plasmodium, cytomegalovirus, herpes simplex virus, and foot and mouth disease virus may be targeted for treatment by introduction of RNA according to the invention. The target gene might be a pathogen or host gene responsible for entry of a pathogen into its host, drug metabolism by the pathogen or host, replication or integration of the pathogen's genome, establishment or spread of an infection in the host, or assembly of the next generation of pathogen. Methods of prophylaxis (i.e., prevention or decreased risk of infection), as well as reduction in the frequency or severity of symptoms associated with infection, can be envisioned.

The present invention also provides methods of identifying gene function in an organism comprising the use of ssRNA and PEI to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceuticals, understanding normal and pathological events associated with development, determining signaling pathways responsible for postnatal development/aging, and the like. The increasing speed of acquiring nucleotide sequence information from genomic and expressed gene sources, including the human genome, can be coupled with the invention to determine gene function for instance in mammalian systems, in particular in human cell culture systems. Putative open reading frames can be determined from nucleotide sequences available in databases using computer-aided searching techniques, as is apparent to one of ordinary skill in the art.

Thus, in another aspect of the invention, a method is provided for assigning function to a DNA sequence, whereby a cell is exposed to PEI and an ssRNA complementary to a desired DNA sequence of unassigned function and in an amount sufficient to inhibit gene expression, identifying a phenotype of the mammalian cell compared to wild type, and assigning the phenotype to the desired nucleic acid.

A simple assay would be to inhibit gene expression according to the partial sequence available from an expressed sequence tag (EST). Functional alterations in growth, development, metabolism, disease resistance, or other biological processes would be indicative of the normal role of the EST's gene product. If database screening finds a region of homology with a protein of known function, a more specific biochemical test based on that function can be used to test for the function of the EST sequence (or inhibition thereof).

The ease with which ssRNA can be introduced into an intact mammalian cell using PEI allows the present invention to be used in high throughput screening (HTS). For example, ssRNA can be chemically synthesized or produced by in vitro transcription.

Solutions containing PEI and ssRNA in an amount sufficient to inhibit a target gene such as, for instance, a differentially expressed gene, can be placed into individual wells positioned on a microtiter plate as an ordered array, and intact cells in each well can be assayed for any changes or modifications in behavior or development due to inhibition of target gene activity or by proteomic, genomics and standard molecular biology techniques. Such a screening is particularly amenable to tissue culture derived from mammals.

A cell that produces a calorimetric, fluorogenic, or luminescent signal in response to a regulated promoter (e.g., transfected with a reporter gene construct) can be assayed in a high throughput format to identify DNA-binding proteins that regulate the promoter. In the assay's simplest form, inhibition of a negative regulator results in an increase of the signal and inhibition of a positive regulator results in a decrease of the signal.

The present invention may be useful in allowing the inhibition of essential genes. Such genes may be required for cell or organism viability at only particular stages of development or cellular compartments. The functional equivalent of conditional mutations may be produced by inhibiting activity of the target gene when or where it is not required for viability.

The invention allows addition ssRNA at specific times of development and locations in the organism without introducing permanent mutations into the target genome. The present invention also provides a kit comprising at least one of the reagents necessary to carry out the in vitro, ex vivo or in vivo introduction of ssRNA using a cationic polymer, in particular PEI as transfection reagent, to test samples or subjects, or construct for its expression for inhibiting expression of a target gene in a mammalian cell. The kit contains a ssRNA and PEI in an amount sufficient to inhibit expression of the target gene, wherein the ssRNA contains a complementary region to the target gene. Such a kit may also include instructions to allow a user of the kit to practice the invention.

The invention is further described, for the purposes of illustration only, in the following examples. Methods of molecular genetics, protein and peptide biochemistry and Immunology referred to but not explicitly described in this disclosure and examples are reported in the scientific literature and are well known to those skilled in the art.

### EXAMPLES

Materials

JetPEI™ was purchased from Polyplus-Transfection (CatNo 101-10). It consists of a linear polymer delivered at a concentration of 7.5 mM (expressed in nitrogen atoms).

Cell Lines

Stably transfected Chinese hamster ovary cells (CHO-K1) (ATCC CCL61, Rockville, Md.) expressing recombinant rat P2X₃ were generated as previously described (Dorn et al. 2001). Cells were cultured in minimal essential medium (MEM-α) supplemented with 10% (v/v) FBS, 2 mM glutamine and 10000 IU/500 ml penicillin/streptomycin in a 5% CO₂-humidified chamber.

Oligonucleotide Synthesis

Oligoribonucleotides for siRNA experiments were synthesized using TOM-phosphoramidite chemistry, as described by the manufacturer (Xeragon) and purified by RP-HPLC. Purity was assessed by capillary gel electrophoresis. Quantification was carried out by UV according to the extinction coefficient at 260 nM.

Annealing of dsRNAs was performed as described elsewhere (Elbashir et. al., 2001). Oligonucleotide sequences are listed below:

| | | |
|---|---|---|
| NAS # | Sequence 5′-3′ | Target |

| | | |
|---|---|---|
| 8646 | 5′ A CUC CAU CCA GCC GAG UGA asg 3′ | P2X₃ sense RNA |
| 8647 | 3′ t stU GAG GUA GGU CGG CUC ACU 5′ | P2X₃ antisense RNA |
| 8549 | 5′ U CGA AGU ACU CAG CGU AAG TT 3′ | unrelated control sIRNA |
| 8548 | 3′ TTA GCU UCA UGA GUC GCA UUC 5′ | |

Abbreviations: N = 2′-H,
n = 2′-O-methoxyethyl,
N =2′-OH,
s =phosphorothioate.

Transfection of CHO-K1 Cells

Polyplexes were prepared immediately prior to transfection. Eighteen hours before transfection, 4×10⁴ cells were plated into 24-well plates in a volume of 0.5 ml MEM-α (supplemented with 10% (v/v) FBS, 2 mM glutamine and 10000 IU/500 ml penicillin/streptomycin) per well. Prior to the transfection, growth medium was removed from the cells and replaced with 500 μl of OptiMEM and 100 μl of the PEI/oligonucleotide mixture. Plates were incubated at 37° in a humidified 5% CO₂ incubator. Subsequently, 60 μl of FBS were added to each well, and the incubation was prolonged for 20 h.

Transfection with jetPEI™

PEI concentration is expressed in nitrogen atom molarity and 1 μg of oligonucleotide contains 3 nmole of anionic phosphate. The volume of linear PEI to be mixed with polynucleic acids in order to obtain the desired N/P (total nitrogen atoms of PEI to the phosphate groups of the oligomers) ratio with regard to the oligonucleotide (ON) concentration was calculated using the following formula:

$$μ I of PEI to be used = \frac{( ON base number ) \times ( pmoles of ON ) \times N \text{/} P ratio \times {10}^{- 3}}{concentration in nitrogen atoms of PEI ( mM )}$$

The same formula was used for siRNA duplexes, considering these reagents as two separate strands.

For 24-well plate experiments carried out in triplicate (final volume of 600 μl in each well), the desired amount of linear PEI was diluted into 150 μl of a 150 mM sterile NaCl solution and then gently vortexed. In a separate Eppendorf tube, the desired amount of oligonucleotide was diluted into 150 μl of a NaCl solution, and then gently vortexed. The 150 μl of PEI solution was then added to the 150 μl nucleic acid solution at once and immediately vortexed for 15 s. The PEI/oligonucleotide solution was left for 15-30 min at RT, then 100 μl of the complex solution were added to each well, containing 500 μl of the desired medium.

RNA Harvesting and Real-Time Quantitative PCR mRNA Analysis

Total RNA was isolated 24 h after oligonucleotide transfection with the RNeasy 96 kit (Qiagen, Chatsworth, Calif.) according to the manufacturers protocol. The RNA samples were individually diluted to 10 ng/μl if a standard from dilutions of pure template mRNA was run, and to 50 ng/12 μl if the mRNA down-regulation was expressed as a percentage of untreated cells. RNA (50 ng loaded for each sample in both cases) was then mixed either with reagents from the real-time quantitative PCR reaction kit PLATINUM Quantitative RT-PCR THERMOSCRIPT One-step System (Invitrogen) or with reagents from the Reverse Transcriptase Q-PCR mastermix kit (Eurogentec) and run according to the included protocol.

Results

The experiments described below were aimed at establishing a simple and universal protocol for applying linear PEI as carrier for the delivery of oligoribonucleotides into mammalian cells. A Chinese Hamster Ovary cell line (CHO-K1) stably transfected with a recombinant rat P2X₃ purinoreceptor cDNA sequence as well as characterized P2X₃ antisense inhibitor sequences were chosen as a model system for siRNA transfections (Dorn et al., 2001).

Single Strand RNA Activity Following Linear PEI Mediated Uptake

Although most antisense compounds used to knock-down mRNA are oligodeoxynucleotides, or contain a 2′-deoxy window (chimeric ASOs) to induce RNase H activity, oligoribonucleotides have also been shown to efficiently trigger mRNA modulation. Endogenous antisense RNA transcripts are present in various organisms to regulate gene expression and have been shown to activate a double strand endoribonuclease which then degrades the target mRNA. Intracellularly expressed antisense RNA constructs have been widely used and, depending on the system, have been shown to induce gene expression inhibition at different levels of RNA processing such as splicing of the primary transcript, transport of the mature mRNA or translation (Pestka et al., 1992). Because of their sensitivity towards nucleases, extracellularly applied short unmodified RNAs do generally not elicit consistent mRNA or protein modulation. Stabilisation of single strand RNA, either through chemical modifications such as phosphorothioate and 2′-modifications in the wings (chimeric RNAs) (Agrawal et al., 1992, Wu et al., 1998) or by hybridisation with a complementary sense strand (Martinez et al. 2002, Schwarz et al. 2002) has then led to active mRNA knock-down reagents.

Having shown that linear PEI is able to transfect various modified ASOs (and particularly the full phosphodiester MOE gapmer, not active when transfected with various lipid formulations) as well as double-stranded RNAs (dsRNA), we further evaluated its protection features against nucleases. Inhibition of P2X₃ mRNA revealed that linear PEI is able to properly transfect, protect and deliver a single strand phosphodiester RNA bearing two MOE DNA modifications and one phosphorothioate linkage at the 3′-end. Whether at 400 nM (table 1) or 200 nM (table 2), the antisense single strand RNA showed an inhibition of the target mRNA of about 50%, whereas the sense single strand RNA was inactive.

TABLE 1
Inhibition of P2X3 mRNA by linear PEI-mediated transfection of
P2X3 single strand (ss) antisense RNA (8647) in CHO-K1 (8646:
single strand sense RNA; 8548/8549: siRNA unrelated control).

| | rP2X3 mRNA level |
|---|---|
| Compound | (% of unrelated double-stranded siRNA) |
| ss antisense P2X3 RNA | 52.8 |
| 8647 (400 nM) | |
| ss sense P2X3 RNA | 107.2 |
| 8646 (400 nM) | |
| Unrelated ds siRNA | 100 |
| 8548/8549 (200 nM) | |

TABLE 2
Inhibition of P2X3 mRNA by linear PEI-mediated transfection
of P2X3 single strand (ss) antisense RNA (8647) in
CHO-K1 (8646: single strand sense RNA)

| | rP2X3 mRNA level |
|---|---|
| Compound | (% of untreated cells) |
| ss antisense P2X3 RNA | 38.0 |
| 8647 (200 nM) | |
| ss sense P2X3 RNA | 74.6 |
| 8646 (200 nM) | |
| Untreated cells | 100 |

### LITERATURE

Agrawal S., Tang J. Y., Sun D., Sarin P. S. and Zamecnik P. C. (1992) Synthesis and anti-HIV activity of oligoribonucleotides and their phosphorothioate analogs. Annals of the New York academy of sciences 660, 2-10
Behr, J. P. (1997) The Proton Sponge—A Trick to Enter Cells the Viruses Did Not Exploit. CHIMIA 51, 34-36.
Boussif, O., Lezoualc'h, F., Zanta, M. A., Mergny, M. D., Scherman, D., Demeneix, B. and Behr, J. P. (1995) A versatile vector for gene and oligonucleotide transfer into cells in culture and in vivo: polyethylenimine. Proc. Natl. Acad. Sci. USA 92, 7297-301
Caplen N. J., Parrish S., Imani F., Fire A., Morgan R. A., (2001) PNAS 98, 9742-9747
Dorn G., Abdel'al S., Natt F., Weiler J., Hall J., Meigel I., Mosbacher J. and Wishart W. (2001) Specific inhibition of the rat ligand-gated ion channel P2X₃ function via methoxyethoxy-modified phosphorothioated antisense oligonucleotides. Antisense & Nucleic Acid Drug Development 11,165-174.
Elbashir S. M., Harborth J., Lendeckel W., Yalcin A., Weber K. and Tuschl T. (2001) Duplexes of 21-nucleotide RNAs mediate RNA interference in cultured mammalian cells. Nature 411, 494-8.
Fire A., Xu S., Montgomery M. K., Kostas S. A., Driver S. E. and Mello C. C. (1998) Potent and specific genetic interference by double-stranded RNA in Caenorhabditis elegans. Nature 391, 806-811
Fischer et al. (1999), Pharm. Res. 16, 1273-1279
Freier S. M. and Altmann K. H. (1997) The ups and downs of nucleic acid duplex stability: structure-stability studies on chemically-modified DNA:RNA duplexes. Nucl. Acids. Res. 25, 4429-4443
D. Hüsken et al, Nucleic Acids Research, 2003, Vol. 31, No. 17 e102
Martin P. (1995) New access to 2′-O-alkylated ribonucleosides and properties of 2′-O-alkylated oligoribonucleotides. Helvetica Chimica Acta 78 (2), 486-504.
Martinez J., Patkaniowska A., Urlaub H., Luhrmann R. and Tuschl T. (2002) Single-stranded antisense siRNAs guide target RNA cleavage in RNAi. Cell 110 (5), 563-574
Pestka S. (1992) Antisense RNA. History and perspective. Annals of the New York academy of sciences 660, 251-262
Schwarz D. S., Hutvágner G., Haley B. and Zamore P. D. (2002) Evidence that siRNAs Function as Guides, Not Primers, in the Drosophila and Human RNAi Pathways. Molecular Cell 10, 537-548
Thompson, J. D. (2002) Applications of antisense and siRNAs during preclinical drug development. Drug Discovery Today 7, 912-916.
Wu H., MacLeod A. R., Lima W. F. and Crooke S. T. (1998) Identification and Partial Purification of Human Double Strand RNase Activity. A novel terminating mechanism for oligoribonucleotide antisense drugs. J Biol Chem 273, 2532-2542.

1. A method for reducing the expression of a target gene comprising exposing a cell to a to a single-stranded oligoribonucleotide and PEI, wherein said single-stranded oligoribonucleotide comprises a region of less than 50 ribonucleotides complementary to the mRNA encoded by said target gene and wherein the target gene is downregulated by RNA interference.

2. A method according to claim 1 wherein said region is less than 25 ribonucleotides.

3. A method according to wherein said region is from 19 to 21 ribonucleotides.

4. A method according to claim 1 wherein said single-stranded oligoribonucleotide consists of the region complementary to the target gene.

5. A method according to the previous claim wherein said cell is a eukaryotic cell.

6. A method according to claim 5 wherein said cell is a mammalian cell.

7. A method according to the previous claim wherein said PEI is linear PEI.

8. A method according to the previous claim wherein the ratio of N/P is between 2 and 10.

9. A method according to claim 8 wherein the ratio of N/P is between 3 and 8.

10. A method according to the previous claim wherein said single-stranded oligoribonucleotides comprise 1, 2, 3, or 4 mismatches and, optionally, wherein 2 to 4 mismatches are adjacent to each other.

11. A method according to claim 1 wherein the single-stranded oligoribonucleotide comprises 1 to 10 chemically modified nucleotides.

12. A method according to claim 11 wherein the chemical modification is a 2′-O-MOE modification.

13. A method according to claim 11 wherein the chemical modification is a modified internucleosidic linkage.

14. A method according to claim 13 wherein the chemical modification is a phophorothioate linkage.

15. A method according to claim 1, wherein the target gene is a human gene.

16. A method according to claim 1 wherein the target gene is a gene that is overexpressed in a pathological condition.

17. A method according to the previous claim wherein the target gene is selected from the following group: oncogene, cytokine gene, viral gene, bacterial gene, development gene, prion gene.

18. Use of linear PEI and a ssRNA for RNA interference.

19. A kit comprising ssRNA and PEI in an amount sufficient to inhibit the expression of a target gene, wherein said ssRNA comprises a region complementary to the target gene and is capable of downregulating said target gene via an RNA interference mechanism.
